(19) United States
(12) Patent Application Publication
(10) Pub. No.: US 2017/0095256 A1
(43) Pub. Date: Apr. 6, 2017

(54) Left Atrial Appendage Occluder

(71) Applicant: Occlutech Holding AG, Schaffhausen (CH)

(72) Inventors: Lars Lindgren, Helsingborg (SE); Rüdiger Ottma, Grossschwabhausen (DE); Sebastian Tilchner, Jena (DE); Michael Heipl, Erfurt (DE)

(73) Assignee: Occlutech Holding AG, Schaffhausen (CH)

(21) Appl. No.: 15/317,087

(22) PCT Filed: Jun. 11, 2015
(86) PCT No.: PCT/EP2015/063011

§ 371 Date: Dec. 7, 2016

Related U.S. Application Data
(60) Provisional application No. 62/010,768, filed on Jun. 11, 2014.

(51) Int. Cl. A61B 17/12

(52) U.S. Cl. CPC A61B 17/12122; A61B 17/12172; A61B 2017/00632

(57) ABSTRACT

An occluder for a left atrial appendage is disclosed comprising a proximal portion comprising a braiding of at least one thread being radially self-expandable in a radial direction, to an expanded state, substantially perpendicular to a surface of inner walls of the left atrial appendage, whereby said braiding is engageable with said inner walls of a proximal end of said left atrial appendage, whereby in said expanded state, said proximal portion has a defined stiffness and resilience to be deformable by, and conformable to, said inner walls, whereby said braiding is configured to form a sealing connection, upon expansion in said radial direction, with said inner walls along a sealing portion of said braiding extending in a longitudinal direction of said occluder, substantially perpendicular to said radial direction. Said occluder comprising further a distal anchoring portion comprising an anchoring wire being radially expandable from a reduced diameter shape to an expanded diameter shape, said anchoring portion having a higher defined stiffness than said proximal portion, and a flexing element connecting said anchoring portion and said proximal portion

### FIELD OF THE INVENTION

This disclosure pertains in general to the field of medical implants. More particularly, the disclosure relates to occluding devices or occluders. In particular the disclosure relates to left atrial appendage occluders and a method of manufacturing thereof.

### BACKGROUND OF THE INVENTION

An occluder is a medical product or implant used for occluding, i.e. closing, defects e.g. in the human heart. Defects may occur in various regions of the heart and have different forms. The occluders can be inserted using minimally invasive cardiac catheter techniques, more precisely by means of a transvenous, catheter-interventional access. One example of a defect is a left atrial appendage (LAA). Thus a left atrial appendage occluder can be placed into such defect in order to prevent thrombus caused by the defect that could transfer to the brain to result in stroke. The occluder is placed into the LAA in order to occlude the LAA and block the blood flow entering the LAA, so that this may eliminate the risk of thrombus. The anatomy of the LAA varies significantly, as seen in FIG. 7 where the LAA defect has been named to reflect the anatomy of the same, e.g. cactus-shape, broccoli-/cauliflower-shape, chicken-wing-shape, and windsock-shape.

FIG. 1 illustrates a prior art disk-type occluder, where the proximal disk covers the LAA opening as a lid.

Prior art LAA occluders are not suitable for all of these type of LAA geometries, and there is a challenge to achieve sufficient occlusion and stability of the occluder over time for the great variance in the LAA geometry.

Thus, it would be advantageous to provide an improved LAA occluder that increases occlusion and have sufficient stability over time, as well as a method of manufacturing such LAA occluder.

### SUMMARY OF THE INVENTION

Accordingly, embodiments of the present disclosure preferably seek to mitigate, alleviate or eliminate one or more deficiencies, disadvantages or issues in the art, such as the above-identified, singly or in any combination by providing an LAA occluder according to the appended patent claims.

According to a first aspect of the disclosure an occluder for a left atrial appendage is disclosed comprising a proximal portion comprising a braiding of at least one thread being radially self-expandable in a radial direction, to an expanded state, substantially perpendicular to a surface of inner walls of the left atrial appendage, whereby said braiding is engageable with said inner walls of a proximal end of said left atrial appendage, whereby in said expanded state, said proximal portion has a defined stiffness and resilience to be deformable by, and conformable to, said inner walls, whereby said braiding is configured to form a sealing connection, upon expansion in said radial direction, with said inner walls along a sealing portion of said braiding extending in a longitudinal direction of said occluder, substantially perpendicular to said radial direction. Said occluder comprising further a distal anchoring portion comprising an anchoring wire being radially expandable from a reduced diameter shape to an expanded diameter shape, said anchoring portion having a higher defined stiffness than said proximal portion, and a flexing element connecting said anchoring portion and said proximal portion and allowing movement between said anchoring portion and said proximal portion from a relaxed configuration to a deployed configuration.

According to a second aspect of the disclosure a method of manufacturing an occluder is disclosed. The method comprises; braiding a tubular or bell-shaped proximal portion of at least one thread; providing a distal anchoring portion comprising a distal anchoring wire and a proximal elongate flexing element; joining said proximal portion and said distal anchoring portion by connecting said flexing element to said proximal portion.

Further embodiments of the disclosure are defined in the dependent claims, wherein features for the second and any other aspects of the disclosure are as for the first aspect mutatis mutandis.

Some embodiments of the disclosure provide for improved anchoring of the occluder in the LAA.

Some embodiments of the disclosure provide for improved anchoring of the occluder in the LAA while maintaining a high flexibility to adapt to various LAA geometries.

Some embodiments of the disclosure provide for an improved occlusion.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the disclosure are capable of will be apparent and elucidated from the following description of embodiments of the present disclosure, reference being made to the accompanying drawings, in which;

FIG. 1 is a prior art occluder;

FIGS. 2a-b illustrates an LAA occluder according to an embodiment of the invention, in a relaxed configuration and a deployed configuration respectively;

FIGS. 3a-b illustrates an LAA occluder according to embodiments of the invention;

FIGS. 4a-f illustrates an LAA occluder according to embodiments of the invention, where FIGS. 4b-e only illustrates the distal anchoring portion thereof for clarity of presentation;

FIG. 5 illustrates an LAA occluder according to an embodiment of the invention;

FIGS. 6a-b illustrates an LAA occluder according to embodiments of the invention;

FIG. 7 illustrates the various anatomies of the LAA;

FIG. 8 illustrates LAA occluders according to embodiments of the invention;

FIG. 9 illustrates LAA occluders according to embodiments of the invention; and

FIG. 10 illustrates a flow chart of a method according to an embodiment of the invention.

### DESCRIPTION OF EMBODIMENTS

Specific embodiments of the disclosure now will be described with reference to the accompanying drawings. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. The terminology used in the detailed description of the embodiments illustrated in the accompanying drawings is not intended to be limiting of the disclosure. In the drawings, like numbers refer to like elements.

The following description focuses on embodiments of the present disclosure applicable to occluders such as an left atrial appendage occluders. However, it will be appreciated that the disclosure is not limited to this application but may be applied to many other medical implants including for example Stents, Vascular Occluders, Products for treatment of aneurysm, Plugs and Occlusion systems for other applications, and various other occluders such as a atrial septal defect (ASD) occluder, a Patent foramen ovale (PFO) occluder, a paravalvular leakage (PLD) occluder, a PDA occluder, a ventricular septal defect (VSD) occluder, or a transapical occluder.

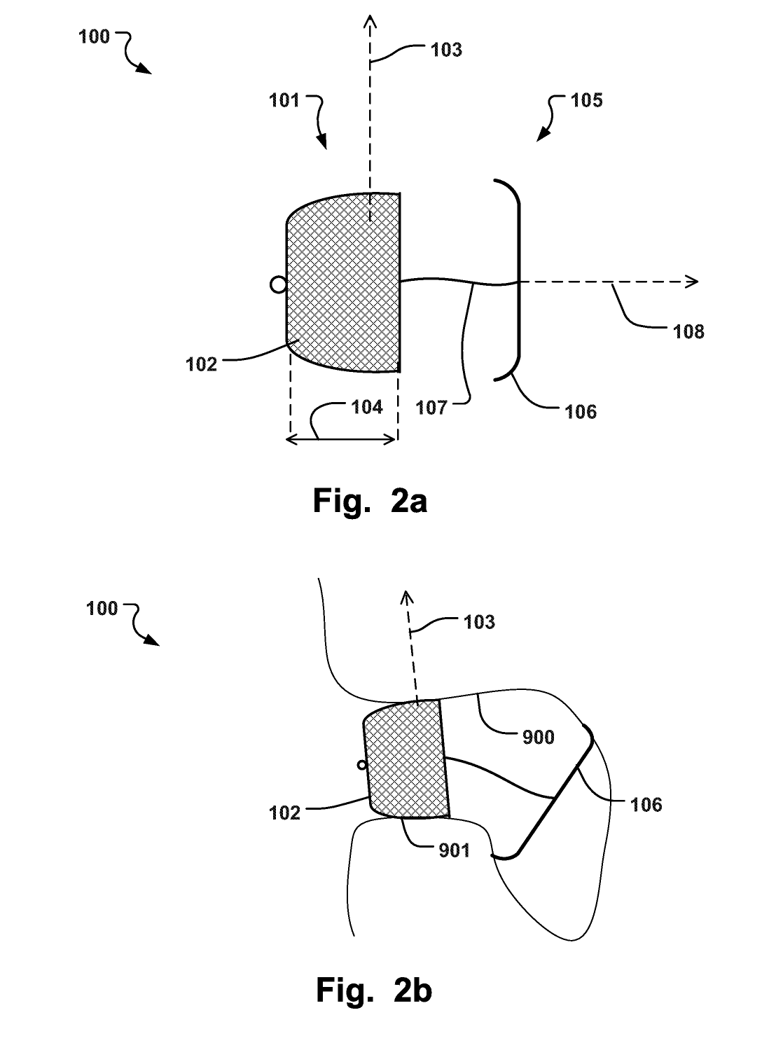

FIGS. 2a-b illustrate an occluder 100 for a left atrial appendage (hereafter referred to as LAA) comprising a proximal portion 101, where the proximal portion comprises a braiding 102 of at least one thread being radially self-expandable in a radial direction 103, to an expanded state, substantially perpendicular to a surface of inner walls 900 of the left atrial appendage (FIG. 2b), whereby the braiding is engageable with the inner walls 900 of a proximal end 901 of the left atrial appendage, and whereby in the expanded state, the proximal portion 101 has a defined stiffness and resilience to be deformable by, and conformable to, said inner walls 900, whereby the braiding 102 is configured to form a sealing connection, upon expansion in the radial direction 103, with the inner walls 900 along a sealing portion 104 of the braiding 102 extending in a longitudinal direction 108 of the occluder 100, substantially perpendicular to the radial direction 103. The occluder further comprises a distal anchoring portion 105 comprising an anchoring wire 106 being radially expandable from a reduced diameter shape to an expanded diameter shape. The anchoring portion has a higher defined stiffness than the proximal portion 101. Stiffness should be construed in the usual meaning, i.e. the extent to which the member resists deformation in response to an applied force. The occluder further comprises a flexing element 107 connecting the anchoring portion 105 and the proximal portion 101 and allowing movement between the anchoring portion 105 and the proximal portion 101 from a relaxed configuration to a deployed configuration.

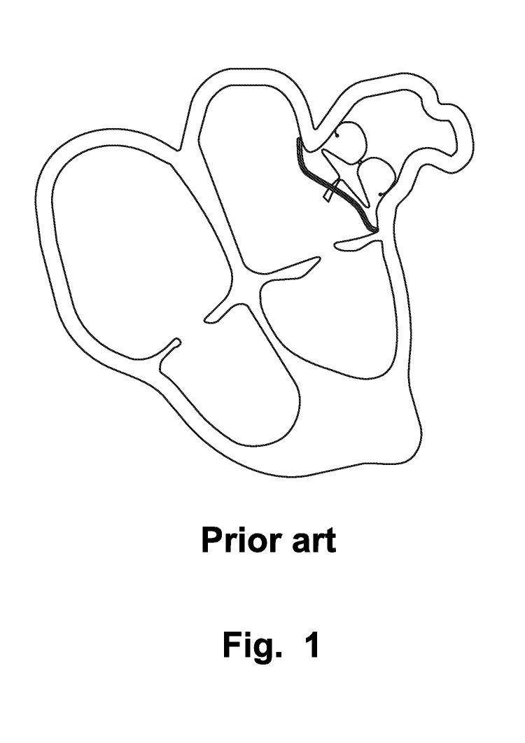

By having a sealing surface 104 of the braiding 102 extending in the longitudinal direction 108 and being configured to expand against the inner walls 900 of a LAA, and being conformable to the inner walls to form a sealing connection thereto, the proximal portion 101 provides an optimal occlusion of the proximal end 901 of a LAA. Independent of the geometry of the proximal portion 101 of the LAA, the proximal portion, i.e. the braiding 102, has sufficiently low stiffness to conform the inner walls 900 of the LAA to form a sealing connection thereto. Prior art devices, such as illustrated in FIG. 1 having a proximal disk relies on the apposition of the proximal disk against the opening of the LAA. Thus the position of such occluder in the longitudinal direction, i.e. how far into the LAA the occluder is positioned, determines if the disk is in contact with the opening or not. Thus the margin of error or tolerances in the positioning for occluding the opening with such disk is low, since is requires accurate positioning in the longitudinal direction for the disk to function as a “lid” over the opening. Eventhough the disk may be correctly positioned at first, there is a significant risk of migration of the occluder due to the forces applied to the occluder from the beating heart. Any migration outwards from the LAA would lead to decerased occlusion or even complete dislodgement of the occluder from the LAA. This in turn requires a more rigid connection to any anchoring portion in order to try to maintain contact between the disk and the LAA opening, i.e. to avoid movement between the anchoring portion and the disk, which also leads to less flexibility between these two portions and thereby less compliance to varying LAA geometries. Further, a proximal occluding disk has the risk of being traumatic due to the movement between the peripheral rims of the disk against the tissue radially outside the opening of the LAA.

Thus the sealing portion 104 of the present embodiments of the occluder 100 extending in the longitudinal direction 108 and configured for expanding radially outwards in the radial direction 103 against the inner walls 900 of the LAA and having a stiffness so as to be deformed by said walls form a sealing connection thereto has improved tolerances in positioning an is less traumatic to the tissue.

Further, the proximal portion 101 may have such sufficiently low stiffness to be conform the inner walls of the LAA due to the distal anchoring portion 105 having a higher defined stiffness than the proximal portion 101. Thus the distal anchoring portion 105 makes sure the proximal sealing portion 101 is fixated in the proximal end 901 of the LAA. Thus, since the proximal portion 101 does not need to provide a counter force to such degree such that it anchors against the inner walls 900—which instead is the function of the distal anchoring portion 105—the proximal portion can have a lower stiffness than the distal anchoring portion 101, and be defined in the heat setting step of the manufacturing thereof to be sufficiently low to be deformed by the force exerted by the inner walls 900 of the LAA to conform and seal thereto.

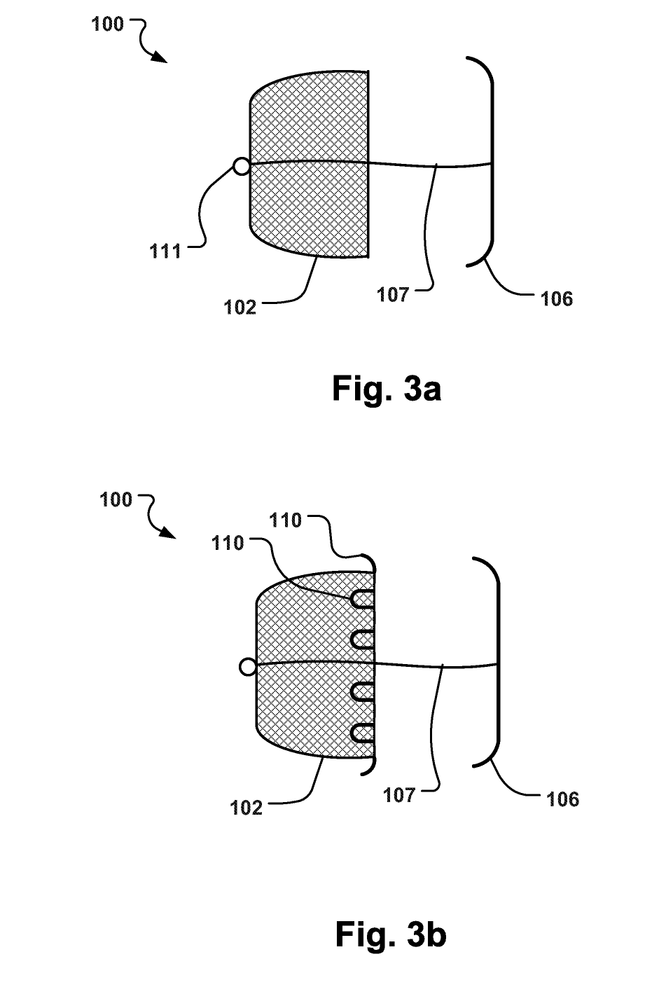

The anchoring wire 106 is radially expandable to an expanded diameter shape, as illustrated in e.g. FIGS. 2a-b, from a reduced diameter shape that fits inside a delivery catheter (not shown). Since substantially the entire distal anchoring portion 105 may be formed by the anchoring wire 106, a very effective tissue anchoring and retention effect can be achieved. The proximal portion 101 may have tissue retention members 110, FIG. 3b, 6b. However, since the anchoring wire 106 is not mounted onto a braiding, and instead being separated as an independent expanding portion of the occluder, independent of the braiding, the degree of tissue retention may be significantly higher due to the expansion force of the anchoring wire itself can be increased of the, in addition to increased stiffness thereof.

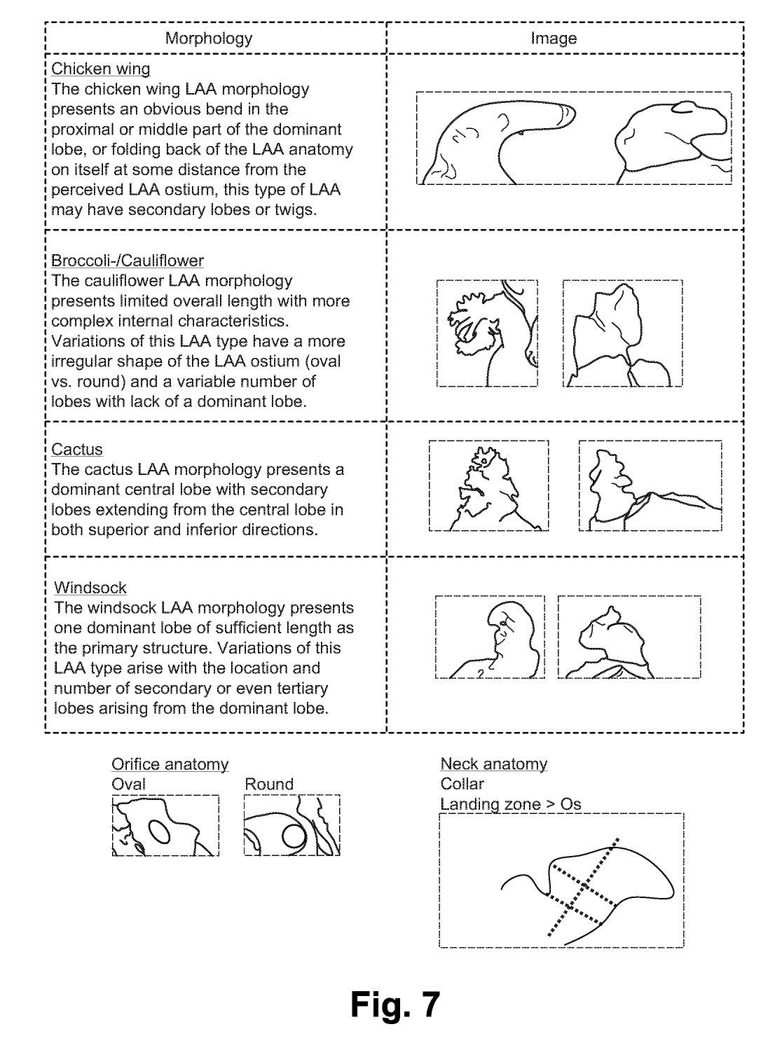

The flexing element 107 of the present embodiments of the occluder 100 allows angling between the proximal portion 101 and a distal anchoring portion 105 for the occluder 100 to adapt to the different geometries of the LAA as seen in FIG. 7. The flexing element 107 may thus be a thin elongate pivotable element that readily allows pivoting between the proximal portion 101 and the distal anchoring portion 105.

The occluder 100 thus provides for the above synergetic features to provide a synergetic effect of being easily conformable to the proximal end 901 of the LAA for sealing thereof, while having a pivotable anchoring portion 105 of higher stiffness than the proximal sealing portion 101 that allows for the proximal portion to be highly conformable, as explained above, while providing secure anchoring in various LAA geometries, such as varying “branches” or “lobes” of the LAA that may be angled relative the entrance opening of the LAA, due to the pivoting flexing element 107 between the two portions.

The braiding 102 of the proximal portion 101 may have a closed or open distal end, i.e. the end opposite connecting unit 111, seen in e.g. FIG. 2a. A braiding that is closed at the distal end may either be provided by having the ends of the at least one thread returning to the connecting unit 111, i.e. a thread may have both ends thereof fixated at the connecting unit 111, e.g. the proximal portion may thus be formed of a bag- or sock-shaped braiding 102. Alternatively, the proximal portion 101 may be formed of a tubular braiding, in which case the ends of the wires thereof can be fixated at both the distal and proximal portion of the braiding 102, such as by a weld cloth at the proximal and distal ends.

The anchoring portion 105 may thus be freely pivotably movable relative the proximal portion, whereby the anchoring portion 105 is positionable at an angle relative the proximal portion, and the longitudinal direction 108, in the deployed configuration, whereby the flexible element 107 is bendable and conformable to the geometry of the left atrial appendage.

The frictional force between the anchoring portion 105 in the expanded diameter shape and the inner walls 900 may accordingly be higher than the frictional force between the proximal portion 101 in the expanded state and the inner walls 900. This improves the anchoring of the proximal portion 101.

The anchoring wire 106 may be stiffer than the at least one thread of the braiding 102. The anchoring wire 106 may thus be of a different material, and/or have a different diameter, and/or have been subjected to a different treatment procedure during manufacturing, than the thread of the braiding 101, in order to be more stiff. Alternatively, or in addition, the geometry of the distal anchoring portion 105 is configured so that the distal anchoring portion 105 has a higher stiffness, e.g. by having a more densely wound structure by the anchoring wire 106

The flexing element 107 may be substantially non-extendable in the longitudinal direction 108 and freely pivotable in the radial direction 103. Thus, this may provide for a substantially fixed separation between the proximal 101 and distal portion 105, while allowing angling therebetween, which may be desired in certain geometries.

The flexing element 107 may comprises a series of linkage section being movable with respect to each other (not shown). This may provide for having a substantially fixed distance between the proximal and distal portions in the longitudinal direction. This may also provede for improved pushability of the occluder while maintaining a high pivoting action.

The flexing element 107 may be resiliently movable in the longitudinal direction and freely pivotable in the radial direction. It may also be desired to have a spring like effect in the longitudinal direction 108, e.g. to provide a tractive force from the distal portion to the proximal portion to draw the latter in a direction further inside the LAA. It may also be desired to have flexibility to reach different branches or lobes of different depths inside the LAA with the distal anchoring portion 105.

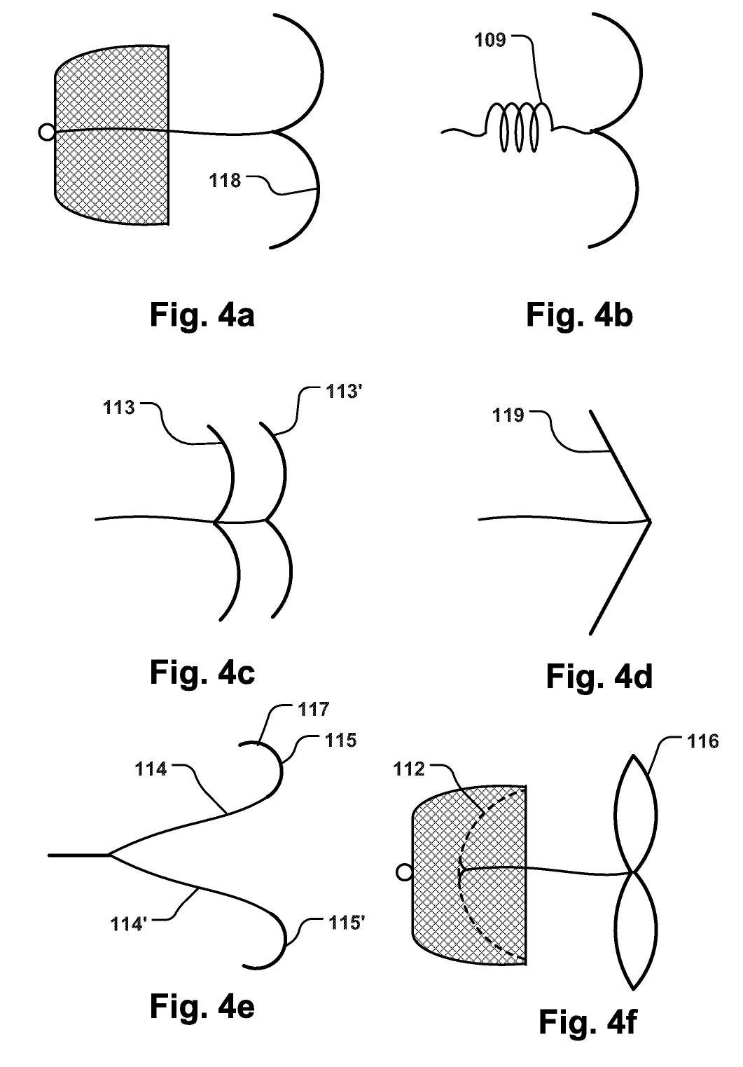

The flexing element 107 may comprises a spring 109 or another resilient element, see FIG. 4b.

The proximal portion 101 may be substantially tubular- or bell-shaped. The sealing portion 104 may extends along substantially the entire length of the proximal portion in the longitudinal direction 108. This provides for a larger sealing interface between the occluder and the inner walls 900 of the LAA, while maintaining a compact device. Also pressure is distributed on a larger surface, as compared to a disk shaped device which is more traumatic.

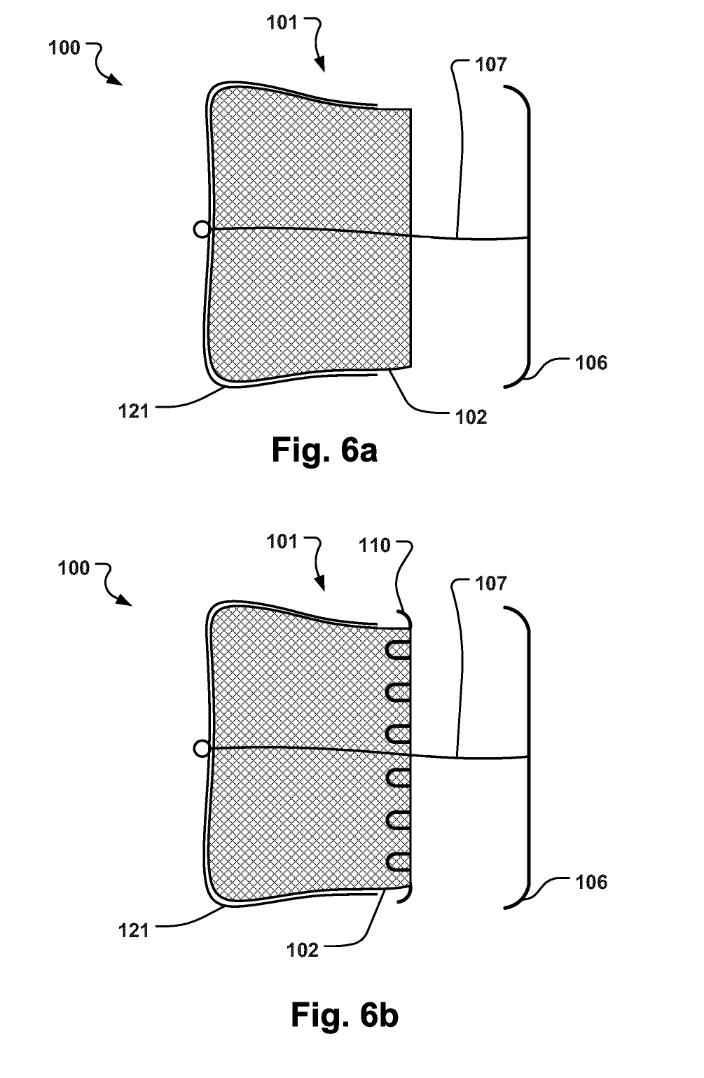

The proximal portion 101 may comprise an occluding membrane 121, as illustrated in FIGS. 6a-b. The membrane may comprise a coating. And the coating may be applied to the occluder 100 by spraying a polymer solution onto the occluder 100. The occluder may be sprayed with a spray which is of a specific viscosity, so that a coating is applied and affixed to an external surface of the occluder. Thus the coating forms covering or membrane over the cells delimited by the threads of the braiding on an external surface of occluder, as illustrated in FIGS. 6a-b. The occluder may be rotated when applying said coating. This may provide for an even and uniform covering over the external surface of the occluder. This may improve the occluding ability and/or biocompatibility. Further, rotating the occluder, allows the coating to be applied to the external surface only. Such selectively may improve on several properties of the implant, such as biocompatibility, structural integrity, flexibility etc. The coating may be made of a biocompatible and implantable material, such as PTE, PTFE or PUR.

The proximal portion 106 may comprise tissue retention members 110, see FIGS. 3b and 6b. Thus may further improve positional stability of the proximal portion 101.

The proximal portion 101 may be resilient such that it is deformable to a non-circular shape by the inner walls 900, such as to an at least partly oval shape, and/or to an at least partly concave shape at a peripheral portion of the proximal portion 101. The stiffness of the proximal portion 101, and/or of the threads thereof, may thus be adapted such that it is readily deformable to a non-circular shape as may be required by the particular geometry of the LAA for optimal sealing.

The occluder may comprise a connecting unit 111 for connecting to a delivery tool, wherein the connecting unit collects and fixates ends of the threads at a proximal end of said the proximal portion 101. The flexing element 107 may be attached to the connecting unit 111 and extend distally therefrom in the longitudinal direction 108, see FIGS. 3a-b, 4a, 6a-b. This allows for ease of manufacturing while providing for the above mentioned advantages of the anchoring portion 105, since no other fixation of the distal portion is necessary.

Alternatively, or in addition, the flexing element 111 may be connected to the braiding 102.

The proximal portion 101 may comprise an outer and inner braiding 112, see FIG. 4f, where the flexing element 107 is connected to the inner braiding 112. This may also allow for longitudinal resiliency between the proximal and distal portions.

The occluder may comprise at least two anchoring portions 113, 113′, see FIG. 4c, being longitudinally displaced in the longitudinal direction 108. This may provide for improved anchoring of the proximal portion 101, e.g. due to more fixation points.

The occluder may comprise at least two flexing elements 114, 114′, see FIG. 4e, being radially displaceable in opposite directions, and each connected to radially opposite anchoring portions 115, 115′. This may allow for anchoring in more than one “branch” or “lobe” of the LAA.

The anchoring portion 105 may comprise at least one wire loop 116, see FIG. 4f, and/or at least one hook 117, see FIG. 4e, and/or at least one barb, and/or at least one tissue piercing member. Various configurations are this possible to provide fixation of the anchoring portion relative the tissue of the LAA.

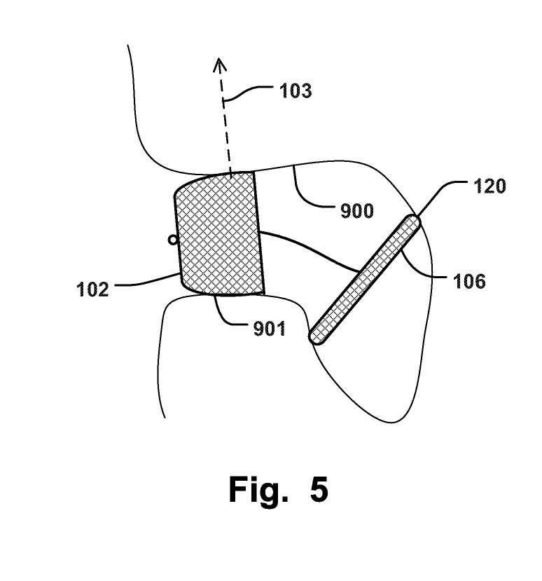

The anchoring wire 106 of the anchoring portion may be braided, i.e. comprising a braid 120, see FIG. 5. The threads of the braid 120 may be stiffer than the threads of the proximal braid 101. Alternatively, the geometry of the braid 120 and/or the thread pattern or configuration of the braid 120 is different from the proximal braid 101 for providing a higher stiffness than that of the proximal braid 101.

The anchoring portion 105 may comprise an at least partly concave shape 118 being concave in a direction towards said proximal portion, see e.g. FIG. 4a. This may improve the retention of the distal portion 105 against the LAA. Alternatively, or in addition, the anchoring portion 105 may comprise an angled portion 119 forming an angle relative the longitudinal direction 108 and in a direction towards the proximal portion 101, see FIG. 4d. this may also improve the retention of the distal portion 105 against the LAA.

The flexing element 107 may be a separate connecting element between the proximal portion 101 and the anchoring portion 106, whereby the flexing element 107 is freely bendable or pivotable independent of, or unaffected by, movement of the threads of the proximal portion 101 and/or movement of the anchoring wire 106 of the anchoring portion 106. This provides for optimal and independent positioning of the anchoring portion 105 relative the proximal portion 101, in the various geometries of the LAA.

As explained above the anchoring wire 106 may also be a separate wire that is unaffected by movement of the threads of the braid of the proximal portion 101, for allowing optimised anchoring function, independent of the movement of the threads of the braiding 102 which move to conform to the internal wall of the LAA for optimal sealing.

The anchoring portion 105 may be radially expandable to a larger diameter than the proximal portion 101. Thus the anchoring of the proximal portion 101 may be further improved.

The distal anchoring portion 105 may comprise the flexing element 107 such that the anchoring wire 106 and the flexing element 107 are joined as an integrated unit. This may provide for a simple to manufacture device while still having the above mentioned advantages.

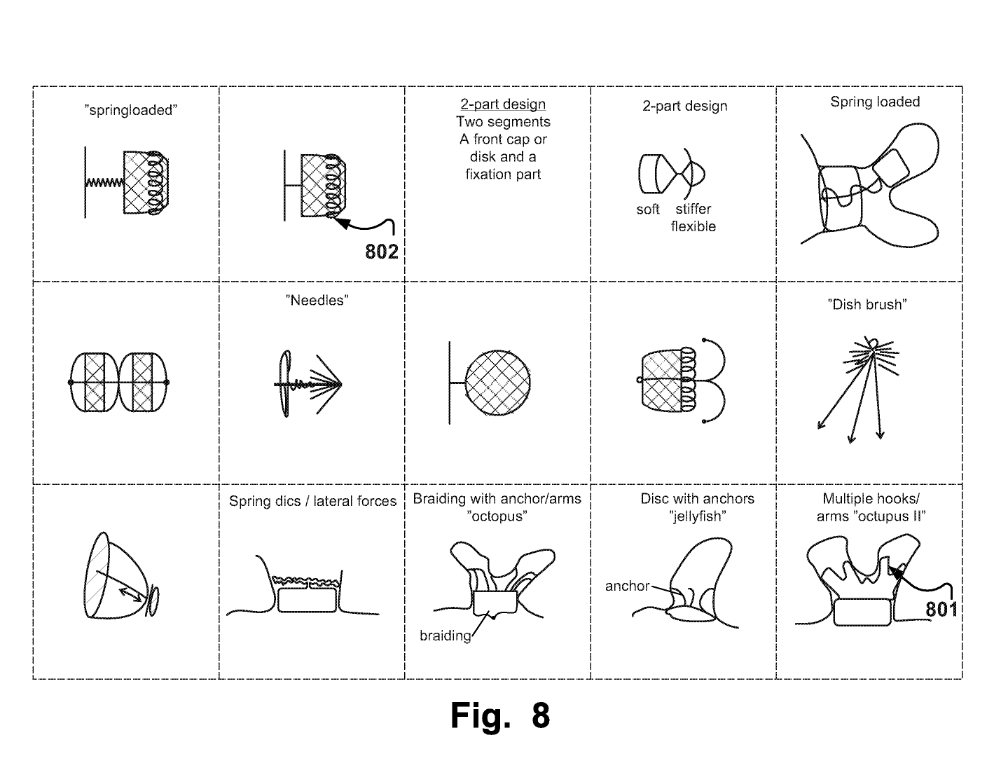

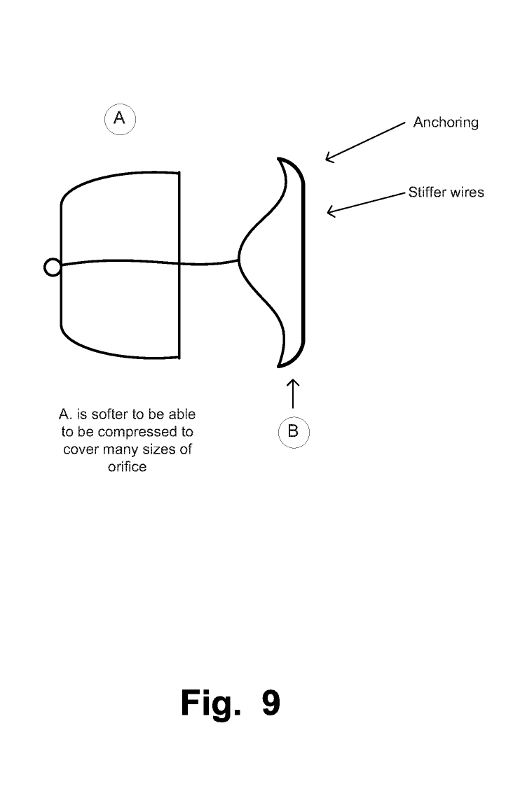

FIG. 8-9 discloses further embodiments of the occluder 100. E.g. FIG. 8 discloses a distal anchoring element 801 comprising an anchoring surface that conforms to the branches and lobes of the LAA, and tissue retention members 802 at the proximal end of the proximal portion 101.

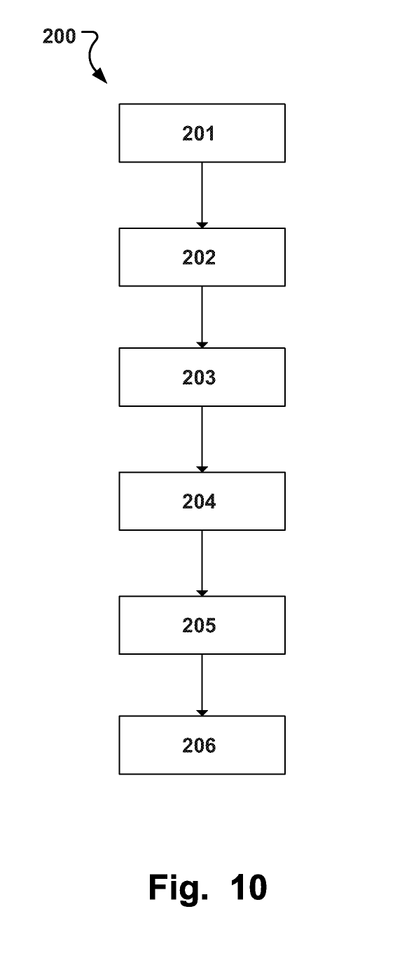

FIG. 10 discloses a method 200 of manufacturing an occluder 100 according to one embodiment is disclosed comprising;

braiding (201) a tubular or bell-shaped proximal portion 101 of at least one thread;

providing (202) a distal anchoring portion 105 comprising a distal anchoring wire 106 and a proximal elongate flexing element 107;

joining (203) said proximal portion and said distal anchoring portion by connecting said flexing element to said proximal portion. This may provide for a particularly easy manufacturing method of the occluder 100 while achieving the above mentioned advantages.

Connecting the flexing element to the proximal portion may comprise;

collecting (204) ends of the at least one thread of said braided proximal portion and an end of said flexing element at a collection point;

attaching (205) said ends of the at least one thread of said braided proximal portion and said end of said flexing element at the collection point.

Attaching said ends of the at least one thread of said braided proximal portion and said end of said flexing element at the collection point may comprise;

welding (206) a connection unit (111) that connects said ends of the at least one thread of said braided proximal portion to said end of said flexing element at the collection point. This provides for a compact and easy to manufacture occluder 100 having the above mentioned advantages.

The present disclosure has been described above with reference to specific embodiments. However, other embodiments than the above described are equally possible within the scope of the disclosure. Different method steps than those described above, may be provided within the scope of the disclosure. The different features and steps of the disclosure may be combined in other combinations than those described. The scope of the disclosure is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

1. An occluder for a left atrial appendage comprising:
a proximal portion comprising a braiding of at least one thread being radially self-expandable in a radial direction, to an expanded state, substantially perpendicular to a surface of inner walls of said left atrial appendage, whereby said braiding is engageable with said inner walls of a proximal end of said left atrial appendage, whereby in said expanded state, said proximal portion has a defined stiffness and resilience to be deformable by, and conformable to, said inner walls, whereby said braiding is configured to form a sealing connection, upon expansion in said radial direction, with said inner walls along a sealing portion of said braiding extending in a longitudinal direction of said occluder, substantially perpendicular to said radial direction,
a distal anchoring portion comprising an anchoring wire being radially expandable from a reduced diameter shape to an expanded diameter shape, said anchoring portion having a higher defined stiffness than said proximal portion,
a flexing element connecting said anchoring portion and said proximal portion and allowing movement between said anchoring portion and said proximal portion from a relaxed configuration to a deployed configuration.

2. Occluder according to claim 1, wherein said anchoring portion is freely pivotably movable relative said proximal portion, whereby said anchoring portion is positionable at an angle relative said proximal portion, and said longitudinal direction, in said deployed configuration, whereby said flexible element is bendable and conformable to the geometry of said left atrial appendage.

3. Occluder according to claim 1, wherein the frictional force between said anchoring portion in the expanded diameter shape and said inner walls is higher than the frictional force between said proximal portion in the expanded state and said inner walls.

4. Occluder according to claim 1, wherein said anchoring wire is stiffer than said thread of said braiding.

5. Occluder according to claim 1, wherein said flexing element is non-extendable in said longitudinal direction and freely pivotable in said radial direction.

6. Occluder according to claim 1, wherein said flexing element is resiliently movable in said longitudinal direction and freely pivotable in said radial direction.

7. Occluder according to claim 1, wherein said flexing element comprises a series of linkage section being movable with respect to each other.

8. Occluder according to claim 1, wherein said flexing element comprises a spring.

9. Occluder according to claim 1, wherein said proximal portion is substantially tubular- or bell-shaped, and wherein said sealing portion extends along substantially the entire length of said proximal portion in said longitudinal direction.

10. Occluder according to claim 1, wherein said proximal portion comprises an occluding membrane.

11. Occluder according to claim 1, wherein said proximal portion comprises tissue retention members.

12. Occluder according to claim 1, wherein said proximal portion is resilient such that it is deformable to a non-circular shape by said inner walls, such as to an at least partly oval shape, and/or to an at least partly concave shape at a peripheral portion of said proximal portion.

13. Occluder according to any of claims 1 12 claim 1, comprising a connecting unit—for connecting to a delivery tool, wherein said connecting unit collects and fixates ends of said threads at a proximal end of said proximal portion, and wherein said flexing element is attached to said connecting unit and extends distally therefrom in said longitudinal direction.

14. Occluder according to claim 1, wherein said flexing element is connected to said braiding.

15. Occluder according to claim 14, wherein said proximal portion comprises an outer and inner braiding, said flexing element is connected to said inner braiding.

16. Occluder according to claim 1, comprising at least two anchoring portions being longitudinally displaced in said longitudinal direction.

17. Occluder according to claim 1, comprising at least two flexing elements being radially displaceable in opposite directions, and each connected to radially opposite anchoring portions.

18. Occluder according to claim 1, wherein said anchoring portion comprises at least one wire loop, and/or at least one hook, and/or at least one barb, and/or at least one tissue piercing member.

19. Occluder according to claim 1, wherein the anchoring wire of said anchoring portion is braided.

20. Occluder according to claim 1, wherein said anchoring portion comprises an at least partly concave shape being concave in a direction towards said proximal portion, and/or an angled portion forming an angle relative said longitudinal direction and in a direction towards said proximal portion.

21. Occluder according to claim 1, wherein said flexing element is a separate connecting element between said proximal portion and said anchoring portion, whereby said flexing element is freely bendable or pivotable independent of, or unaffected by, movement of the threads of said proximal portion and/or movement of the anchoring wire of said anchoring portion.

22. Occluder according to claim 1, wherein said anchoring portion is radially expandable to a larger diameter than said proximal portion.

23. Occluder according to claim 1, wherein said distal anchoring portion comprises said flexing element such that said anchoring wire and said flexing element are joined as an integrated unit.

24. A method of manufacturing an occluder according to one embodiment is disclosed comprising:
braiding a tubular or bell-shaped proximal portion of at least one thread;
providing a distal anchoring portion comprising a distal anchoring wire and a proximal elongate flexing element;
joining said proximal portion and said distal anchoring portion by connecting said flexing element to said proximal portion.

25. Method according to claim 24, wherein connecting the flexing element to the proximal portion comprises:
collecting ends of the at least one thread of said braided proximal portion and an end of said flexing element at a collection point;
attaching said ends of the at least one thread of said braided proximal portion and said end of said flexing element at the collection point.

26. Method according to claim 25, wherein attaching said ends of the at least one thread of said braided proximal portion and said end of said flexing element at the collection point comprises:
welding a connection unit that connects said ends of the at least one thread of said braided proximal portion to said end of said flexing element at the collection point.
